(19) United States
(12) Patent Application Publication
(10) Pub. No.: US 2023/0414141 A1
(43) Pub. Date: Dec. 28, 2023

(54) SYSTEMS AND METHODS FOR MONITORING MENTAL STATE

(71) Applicant: MINDWELL LABS INC., New York, NY (US)

(72) Inventors: Shaun S. NANAVATI, New York, NY (US); Saurabh KANAUJIA, New York, NY (US)

(73) Assignee: MINDWELL LABS INC., New York, NY (US)

(21) Appl. No.: 18/037,479

(22) PCT Filed: Nov. 18, 2021
(86) PCT No.: PCT/US2021/059817

§ 371 Date: May 17, 2023

Related U.S. Application Data
(60) Provisional application No. 63/115,172, filed on Nov. 18, 2020.

(51) Int. Cl. A61B 5/16; A61B 5/024; A61B 5/00; A61B 5/318

(52) U.S. Cl. CPC A61B 5/165; A61B 5/02405; A61B 5/746; A61B 5/681; A61B 5/318

(57) ABSTRACT

Disclosed are systems and methods for measuring and monitoring mental state of a user. In some examples, these systems and methods may calculate a user's mental state based on variation in heart rate data. The system may provide content and programming to the user based on their mental state.

### CROSS-REFERENCE TO RELATED APPLICATIONS

This application claims benefit under 35 U.S.C. § 119(e) of U.S. Provisional Application No. 63/115,172, filed Nov. 18, 2020, content of which is incorporated herein by reference in its entirety.

### FIELD

The present invention is directed to systems and methods for measuring the mental state a user.

### BACKGROUND

The following description includes information that may be useful in understanding the present invention. It is not an admission that any of the information provided herein is prior art or relevant to the presently claimed invention, or that any publication specifically or implicitly referenced is prior art.

Existing anxiety monitors are administered by medical experts using clinical grade equipment. Therefore, anxiety monitors are generally administered at a single point of time at the point of care in clinical settings.

### SUMMARY

Disclosed are systems and methods for continuously monitoring the mental state of user and providing feedback and programming to address the mental state of necessary. The term “mental state,” as used herein, may refer to a state of mind that is anxious, focused, calm, neutral, vigilant, relaxed, or related states of mind. For instance, in some examples, the disclosed systems and methods determine a user's state of mind (e.g., cognitive arousal) on a sliding scale from anxious (i.e., vigilant, hyper, stressed, etc.), to neutral (i.e., calm/relaxed, etc.), to focused. In some examples, the mental state will be discrete categories based on thresholds. In other examples, the mental state may be a continuous scale to provide a cognitive arousal score.

As described herein, the systems and methods may process heart rate data to determine the mental state. The model for processing the heart rate data may process previously recorded heart rate data in certain time windows to determine a deviation from a baseline heart rate. The system may continuously monitor the mental state and heart rate data to provide an ongoing determination of mental state.

In some examples, the system uses a wearable (e.g., a smartwatch) that monitors heart rate data to infer changes in cognitive arousal. This is in contrast to existing systems that measure arousal using EEG and require electrodes to be applied to the head.

Accordingly, the systems and methods herein will provide user self-awareness of their mental state on a continuous basis and allow for tracking and trend monitoring. Self-awareness of changes in anxiety and focus on a real-time and 24/7 basis can then be utilized to determine what triggers and factors in the environment at any point could be causing individual anxiety or focus. In some examples, the disclosed technology may identify text, voice, video or other calls that trigger anxiety or focus as an application may monitor the mental state while the user is utilizing a mobile device for other tasks that may be monitored and correlated to mental state.

Understanding changes throughout the day can also be helpful in creating a treatment approach that is based upon delivery of content through an application as needed at that specific moment, rather than treating it as a ubiquitous phenomenon. In some examples, the disclosed technology may utilize a feedback loop to identify optimal treatments for specific users. For instance, sound therapy with particular songs, frequencies, and amplitudes may be delivered to the user at specific time and the technology may be able to determine which types of sound therapy deliver the fastest and largest improvement in mental state. In other examples, the system may monitor the user's environment (e.g. temperature, lighting) including based on integration from smart home lights, switches and thermometers. For instance, the application may change the user's light and temperature as an output form the disclosed technology if, for instance, smart applications are integrated with the disclosed application.

In some examples, the system processes heart rate data to determine the user's mental state. In some examples, this may include determining whether the user is: (1) anxious, (2) focused, or (3) neutral based on a deviation of the current heart rate from a baseline heart rate. Following are example threshold rules for determining various mental states, however different rules may be determined, including based on continuous rather than discrete categories.

Anxiety: in some examples, an anxious mental state may be identified by a rise in the user's heart rate of 15, 16, 17, 18, 19, 20, 21, 22, 23, 24, or 25 percent (or other suitable threshold percentages) within a 15, 16, 17, 18, 19, 20, 21, 22, 23, 24, or 25 minute (or other suitable time frames) period from a baseline (neutral/calm) state that was identified at least 20 minutes ago or longer.

Focus: in some examples, a focused mental state may be identified by a drop in the user's heart rate of 5, 6, 7, 8, 9, 10, 11, 12, 13, 14, or 15 percent (or other suitable threshold percentages) within a 15, 16, 17, 18, 19, 20, 21, 22, 23, 24, or 25 minute period (or other suitable time frames) from a baseline (neutral/calm) state that was identified at least 20 minutes ago or longer.

Neutral: in some examples, a neutral state may be defined as neither anxious or focused state and could be based on an average heart rate for a prior time period (e.g. prior day, week, month, etc.). In some examples, the time windows and thresholds may be updated every time a data feed containing heart rate data is updated or received by the system. For instance, this could be every 1, 2, 3, 4, 5, 10, 15, 30, or 45 seconds or 1, 2, 3, 4, 5, 6, 7, 8, 9, or 10 minutes.

### BRIEF DESCRIPTION OF THE DRAWINGS

The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1 depicts an example of an overview of a system for implementing the disclosed technology.

FIG. 2 depicts a flow chart showing example processes for implementing the disclosed technology.

FIG. 3 depicts a flow chart showing an example process for implementing the disclosed technology.

FIG. 4 depicts an example of chart showing a data readout of the disclosed technology.

In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced.

### DETAILED DESCRIPTION

Unless defined otherwise, technical and scientific terms used herein have the same meaning as commonly understood by one of ordinary skill in the art to which this invention belongs. Szycher's Dictionary of Medical Devices CRC Press, 1995, may provide useful guidance to many of the terms and phrases used herein. One skilled in the art will recognize many methods and materials similar or equivalent to those described herein, which could be used in the practice of the present invention. Indeed, the present invention is in no way limited to the methods and materials specifically described.

In some embodiments, properties such as dimensions, shapes, relative positions, and so forth, used to describe and claim certain embodiments of the invention are to be understood as being modified by the term “about.”

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations may be depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

### Mental State Monitoring Systems

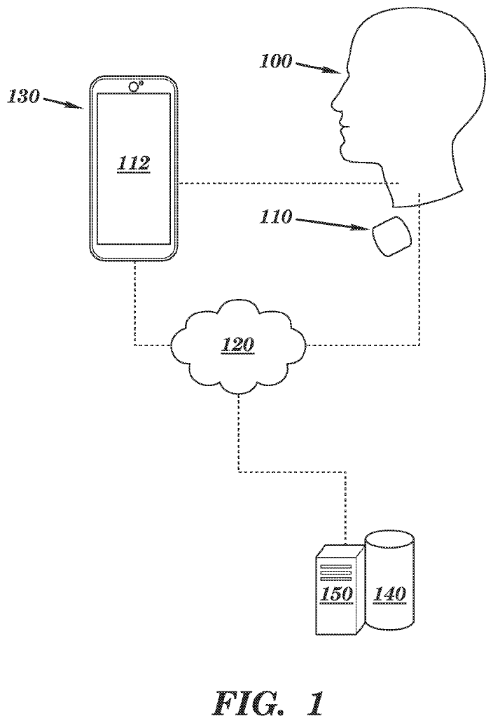

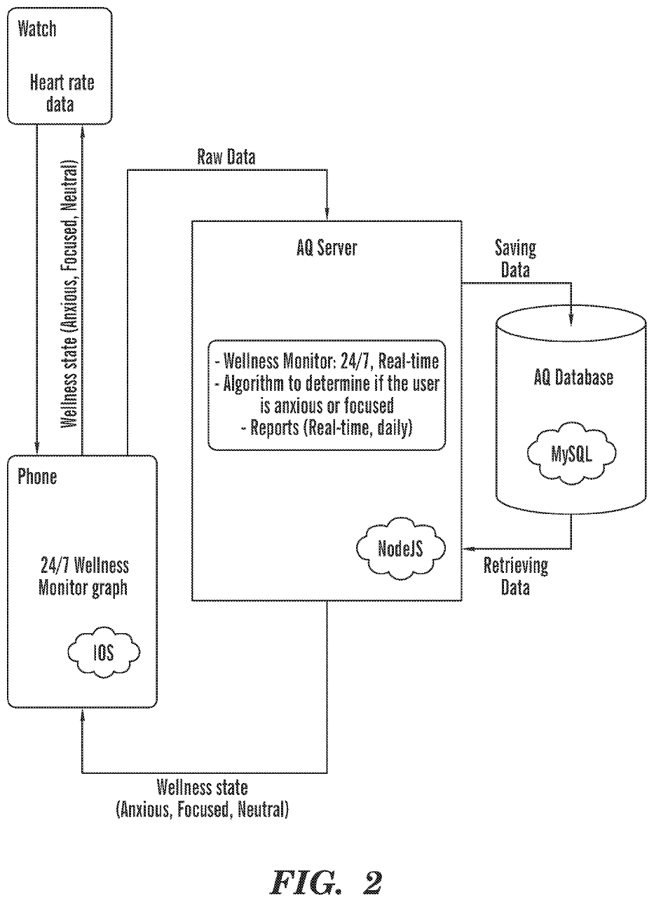

FIG. 1 illustrates an example system for implementing the disclosed technology. For instance, the system may contain a computing device 130 with a display 112, a network 120, a patient 100, a sensor 110, a server 150, and database 140. The computing device 130 may be any suitable computing device, including a computer, laptop, mobile phone, etc. The network 120 may be wired, wireless, or various combinations of wired and wireless. The server 150 and database may be local, remote, and may be combinations of servers 150 and databases 140, or could be local processors and memory.

The sensor 110 may be a smart phone, smart watch, smart ankle bracelet, smart glasses, smart ring, patch, band, or other device that suitably could be retained on the patient 100 and output heart rate data from the patient. In other examples, the wearable 110 may be a clinical grade ECG system. In some examples, the sensor 11 may be a camera on a mobile device and may record the heart rate data by fluctuation in colors of the capillaries detected by the camera.

FIG. 2 illustrates an overview of an example system for monitoring mental state and delivering programming to the user to address mental state. Accordingly, as illustrated, a server may execute various algorithms and models utilized to determine mental state, and display the mental state to the user in various formats. In some examples, the system may send content recommendations to the user to address the mental state. These content recommendations may be uniquely personalized for each user based on their mental state and historical data on effectiveness of content as described herein.

In some examples, the system may simultaneously record contextual data received by an application associated with the user's mobile device or other hardware. For instance, the application may receive information on the user's engagement with various applications and third parties on the mobile device. This may include who the user is communicating with through text, email, voice calls, video calls, and accordingly may be able to associate anxious states with particular activities or contacts. For instance, the system may tag anxious, focused or calm states determined at a certain time with additional information that includes:
(1) Conversations referenced to a particular contact;
(2) Modality of conversation (text, email, voice, video, etc.);
(3) Time of day;
(4) Music tracks playing;
(5) Temperature;
(6) Lighting through integration with smart home devices;
(7) Social medial usage; and
(8) Others.

The mental state may be determined from heart rate data. The heart rate data may be output from a smart watch with ECG capabilities and sent to a user's mobile device/smartphone. The user's mobile device may include an application that may provide content recommended for the user to improve their mental state and reports showing the user's mental state—including the current mental state and trends of the mental state over time (e.g. daily, weekly, monthly).

In some examples, raw heart rate data may be processed on a user's smart watch, on a user's smart phone/mobile device, or may be sent in raw format to a server where the mental state algorithms are stored for processing. A server may include a database connected to the server that includes mental state data stored from the user and third party users.

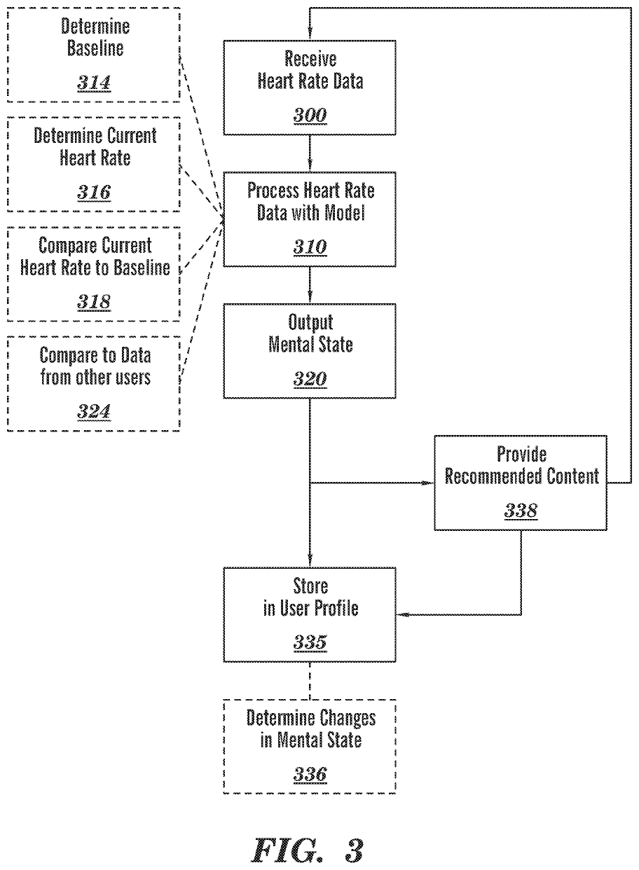

FIG. 3 illustrates an example process for determining mental state and delivery content to a user. For instance, heart rate data may be received 300 from a smart watch, or a mobile device with a camera after a user presses their finger over the camera sensor. However, in most embodiments, the system will be able to passively monitor the heart rate without requiring user action. Accordingly, the technology may receive heart rate data on a periodic basis and may be passively received (without requiring user interaction with the application or devices). For instance, this could be could be every 1, 2, 3, 4, 5, 10, 15, 30, or 45 seconds or 1, 2, 3, 4, 5, 6, 7, 8, 9, or 10 minutes. Then, the heart rate data may be processed with a model 310 to determine the mental state including by determining the current heart rate 316.

For instance, the model may process the heart rate data to compare the current heart rate with a baseline heart rate associated with a neutral or calm mental state. This allows the system to determine whether any current deviation of the current heart rate from the baseline is within a threshold that would indicate the user is no longer calm and either focused (if there is a significant enough decrease in heart rate) or anxious (if there is a significant enough increase in heart rate). For instance, every time the system receives a new set of heart rate data 300, it may determine a new baseline heart rate based on previously recorded/received heart rate data based on the updated time frames and windows. Then, the current heart rate (and/or trends in heart rates) may be used to determine the current mental state by comparison to the baseline heart rate.

In some examples, the baseline 314 may by identified as the last heart rate recorded longer than 20 minutes ago that was not determined to be an anxious or focused state or within a threshold time window of an anxious or focused state. For instance, the system may process previously recorded heart rate data to identify a heart rate reading staring at a threshold of at least 15, 16, 17, 18, 19, 20, 21, 22, 23, 24, or 25 minutes ago, or other suitable time frame in the past, when the system determined the user's mental state was neutral/calm (not focused or anxious) and the reading was not within 2, 3, 4, 5, 6, 7, 8, 9, or 10 minutes (or other suitable time frames) of a future anxious reading.

In some examples, the baseline 314 may be an average heart rate from a previous time window (e.g. a previous day's, week's, or month's heart rate). In some examples, the baseline 314 may be a user assigned heart rate when the user is calm (e.g. by tagging a heart rate or indicating through an interface on the application that the user is calm). In some examples, the baseline may be an average of a rolling time window of 1, 2, 3, 4, 5, 6 hours or other suitable time frames.

Accordingly, the model may determine a percentage deviation (or other quantification of the deviation) of the current heart rate from the baseline, and use predefined threshold or percentages to determine the current mental state. For instance, in some examples, the system may determine the user is anxious if the current heart rate is 15, 16, 17, 18, 19, 20, 21, 22, 23, 24, or 25 percent greater than the baseline heart rate. In some examples, the system may determine the user is focused if the current heart rate is 5, 6, 7, 8, 9, 10, 11, 12, 13, 14, or 15 percent lower than the baseline heart rate.

Next, the system may output the mental state 230 (e.g. display in various formats, including with historical data) and store the mental state and details in a user profile 335 associated with the user. This may include a date and time stamp for the mental state and any other contextual information including day of the week. In some examples, the system may store multiple mental states for a user and then determine changes in the mental state 336, which may include trends or associations with other contextual information.

After determining the mental state, the system may then provide recommended content to the user 338 to improve the mental state or recommendations based on context. The content may include training lessons, meditations, breathing exercise, physical movements, sound therapy, change in user's environment (room temperature and lighting), recommendations to end conversations with contact, recommendations to reach out to a list of contacts, etc. In some examples, every time the user completes content delivered to the user through the system, the system may store the content session completion information in the user profile 335.

Accordingly, the system may provide recommended content based on changes in the mental state, or specific components/measures of mental state after delivery of content. For instance, the system may deliver sound therapy through a speaker of a device controlled by an application as disclosed herein using various combinations of: (1) songs, (2) volumes, (3) amplitudes, (4) frequencies, (5) tempos, (6) genres, and may simultaneously record heart rate data and determine the user's mental state.

Accordingly, the system may record and monitor the above aspects of the sound therapy (or other content) that improve the mental state by the most and/or the fastest and have the longest impact on mental state long term. Accordingly, this will allow the system to proactively recommend to the user that they listen to certain sounds or tones. In some examples, the system may modulate the frequencies of sound therapy while a user is listening based on feedback from their mental state. For instance, the amplitude of certain frequencies may be increased on a particular song or sound therapy file by accessing the sound mixer of the hardware device delivering the sound therapy.

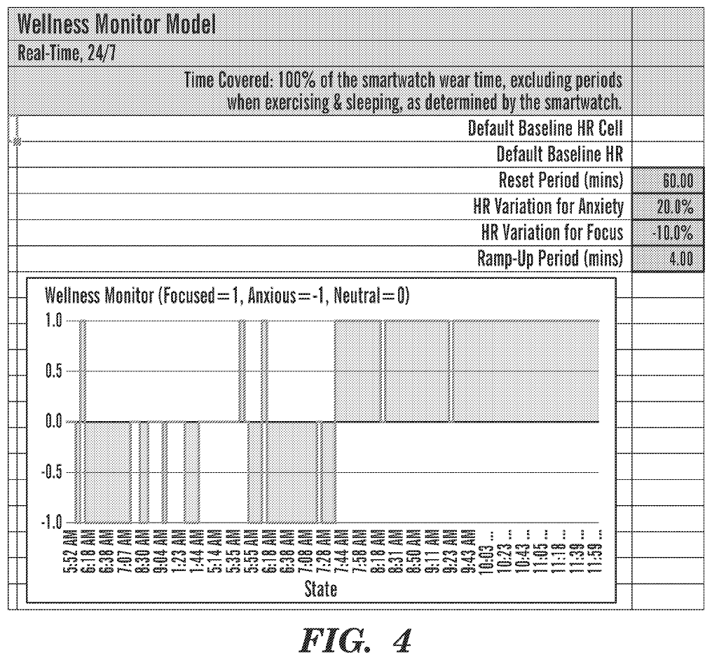

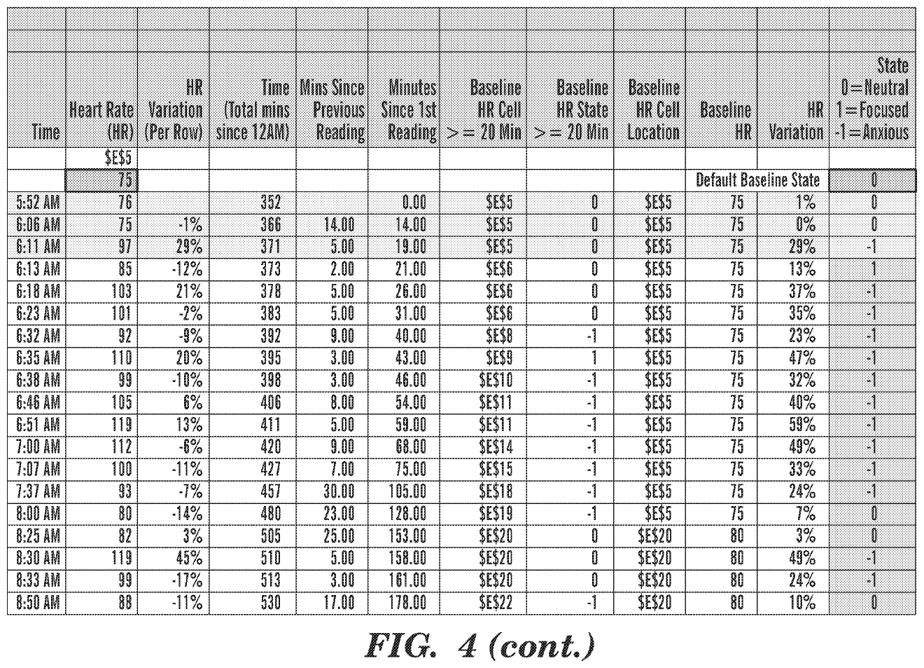

FIG. 4 illustrates an example data feed from a user that illustrates heart rates determined at various points in time, the determined baseline, the deviation from the baseline of the current heart rate, and the determined mental state. Additionally, FIG. 4 illustrates an output of the mental state over a day in a graph format.

### Computer & Hardware Implementation of Disclosure

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

### CONCLUSION

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Certain embodiments of this application are described herein. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

Particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

1. A system for monitoring attention, the system comprising:
a sensor for outputting heart rate data;
a memory containing machine readable medium comprising machine executable code having stored thereon instructions for performing a method;
a control system coupled to the memory comprising one or more processors, the control system configured to execute the machine executable code to cause the control system to:
receive a set of heart rate data representing a user's current heart rate;
determine a baseline heart rate based on previously recorded heart rate data for the user;
process the set of heart rate data and the baseline heart rate to output an indication of mental state; and
store the indication of mental state referenced to a unique identifier referenced to the user in the memory.

2. The system of claim 1, wherein the control system is configured to execute the machine executable code to further cause the control system to send a notification to a user interface associated with the user with a set of content based on the mental state.

3. The system of claim 1, wherein the mental state is an estimate of at least one of calm, focus or anxiety.

4. The system of claim 1, wherein the set of heart rate data is continuously acquired from the sensor.

5. The system of claim 5, wherein the control system is configured to execute the machine executable code to further cause the control system to send a notification to an interface associated with the user if the indication of mental state changes.

6. The system of claim 2, wherein the set of content comprises at least one of: training lessons, meditations, breathing exercise, physical movements, sound therapy, or changes in the patient's room temperature or lighting.

7. The system of claim 8, wherein the control system is configured to execute the machine executable code to further cause the control system to store to the memory a number and type of the set of content performed through the interface by the user.

8. The system of claim 1, wherein the sensor is an ECG sensor on a smart watch.

9. The system of claim 1, wherein the sensor is a camera on a mobile device.

10. The system of claim 1, wherein the control system is configured to execute the machine executable code to further cause the control system to receive a second set heart rate data from a second sensor associated with a second user.

11. The system of claim 1, wherein process the set of heart rate data comprises determining whether the heart rate changes a threshold percentage within a sliding time window.

12. The system of claim 1, wherein process the set of heart rate data further comprises determining whether the heart rate is a threshold amount greater than the baseline heart rate in a sliding time window.

13. The system of claim 1, further comprising:
receiving a second set of heart rate data; and
determining a second baseline heart rate based on the second set of heart rate data and previously recorded heart rate data comprising the first set of heart rate data; and
processing the second set of heart rate data, the second baseline heart rate and the first set of heart rate data to determine a second indication of mental state.

14. The system of claim 1, wherein the baseline comprises a historical heart rate of the user recorded while the user was not anxious or focused that was within a time window.

15. The system of claim 14, wherein the time window is greater than twenty minutes from the current time.

16. The system of claim 1, wherein the baseline heart rate comprises an average of a previous time period's heart rate readings.

17. The system of claim 16, wherein the previous time period is a previous day.

18. The system of claim 1, wherein the baseline heart rate comprises a rolling average heart rate of a sliding time window.

19. The system of claim 1, wherein the baseline heart rate comprises the last previously recorded heart rate that was not classified as anxious or focused, was greater than a threshold time in the past, and was not within a threshold time window of a heart rate reading classified as anxious or focused.

20. The system of claim 1, wherein process the set of heart rate data using the baseline heart rate to output an indication of mental state comprises calculate a percentage change of a current heart rate from the baseline heart rate.

21. The system of claim 20, wherein the indication of mental state comprises anxious if the percentage change is an increase of twenty percent.

22. The system of claim 20, wherein the indication of mental state comprises focused if the percentage change is a decrease of twenty percent.

23. The system of claim 1, wherein the set of content is selected from a set of sound files that each comprise a set of features.

24. The system of claim 23, wherein the set of features comprises a set of frequencies and a set of amplitudes.

25. The system of claim 24, wherein the content is selected based on previous data indicating a change in the user's mental state after playing at least one sound file in the set of sound files.

26. A system for monitoring attention, the system comprising:
a sensor for outputting heart rate data;
a memory containing machine readable medium comprising machine executable code having stored thereon instructions for performing a method;
a control system coupled to the memory comprising one or more processors, the control system configured to execute the machine executable code to cause the control system to:
receive heart rate data representing a user's heart rates at regular time intervals;
determine a mental state of the user at each of the regular time intervals by comparing the user's current rate to historical heart rate data; and
store an indication of the mental state at each time interval.
